(19) United States
(12) Patent Application Publication
(10) Pub. No.: US 2026/0253144 A1
(43) Pub. Date: Aug. 27, 2026

(54) SYSTEMS AND METHODS FOR USING TRAINED PREDICTIVE MODELING TO REDUCE MISDIAGNOSES OF CRITICAL ILLNESSES

(71) Applicant: Evernorth Strategic Development, Inc., St. Lois, MO (US)

(72) Inventors: Daniel J. Greden, West Hartford, CT (US); David Spangler, Hilton Heard, SC (US); Bhuvana Sagar, Spring, TX (US); Richard S. Emery, Portland, OR (US); Stephen A. Hamilton, Edmond, OK (US); Eric J. Gratias, Flagler Beach, FL (US)

(21) Appl. No.: 19/652,629

(22) Filed: Apr. 20, 2026

Related U.S. Application Data
(63) Continuation-in-part of application No. 17/462,169, filed on Aug. 31, 2021.
(60) Provisional application No. 63/072,605, filed on Aug. 31, 2020.

(51) Int. Cl. G06Q 40/08; G16H 10/60; G16H 20/13; G16H 40/67; G16H 50/20; G16H 50/70

(52) U.S. Cl. CPC G06Q 40/084; G06Q 40/09; G16H 10/60; G16H 20/13; G16H 40/67; G16H 50/20; G16H 50/70

(57) ABSTRACT

A trained predictive server is provided for determining that a diagnosis and treatment plan is inaccurate. The trained predictive server includes a processor configured to receive a set of prior authorization (PA) data associated with a medical claim for a patient, and determine that the PA data indicates that the medical claim is associated with a qualifying critical illness and control use of medical devices. The processor can extract component data from the set of PA data, and apply the extracted component data to a predictive model associated with the qualifying critical illness to determine whether the medical claim is associated with an inaccurate diagnosis and treatment plan. Upon determining that the medical claim is associated with an inaccurate diagnosis and treatment plan, the processor can generate a request for review of the diagnosis and treatment plan using the set of PA data and stop use of a medical device.

### CROSS-REFERENCE TO RELATED APPLICATION

This application claims the benefit of U.S. patent application Ser. No. 17/462,169, filed Aug. 31, 2021, as a continuation-in-part, which claims benefit to U.S. Provisional Patent Application No. 63/072,605, filed Aug. 31, 2020, both of which are incorporated by reference herein in there entirety.

### FIELD

The field relates to systems and methods for using trained predictive models to predict whether diagnoses of critical illnesses are inaccurate and require a second opinion. The field also relates to the use of prior authorization (PA) data and related systems to facilitate the training of such predictive models.

### BACKGROUND

Many known critical illnesses, including chronic illnesses and terminal illnesses, are susceptible to misdiagnosis. There are many reasons for such misdiagnoses. Such critical illnesses are being actively researched and studied, making accurate assessment of the illnesses difficult to determine for healthcare providers who are not actively following the state-of-the-art related to each illness. Further, many such critical illnesses are rare and/or complex and make it difficult for even some specialist healthcare providers to readily understand. Additionally, reliable diagnosis of such critical illnesses may depend upon the availability of testing and diagnostic tools that may not be available to all healthcare providers.

At least some misdiagnoses may lead to adverse outcomes, including death. For example, failure to accurately diagnose an illness could delay or impede the creation, development, and/or implementation of an appropriate treatment plan. Left untreated, at least some illnesses tend to get worse over time. Moreover, at least some misdiagnoses could result in a treatment plan that is ineffective or even counterproductive. Further, because treatment can be expensive and resource intensive, such misdiagnoses may contribute to unnecessary waste of medical resources and financial resources of patients and insurers.

### BRIEF SUMMARY

Examples described herein enable predicting whether diagnoses or treatments of critical illnesses are inaccurate. In one aspect, a trained predictive server is provided for determining that a diagnosis and treatment plan is inaccurate. The trained predictive server includes a processor and a memory. The processor is configured to receive a set of prior authorization (PA) data associated with a medical claim for a patient, and determine that the set of PA data indicates that the medical claim is associated with a qualifying critical illness. The processor is further configured to extract component data from the set of PA data, and apply the extracted component data to a trained predictive model associated with the qualifying critical illness to determine whether the medical claim is associated with an inaccurate diagnosis and treatment plan. Upon determining that the medical claim is associated with an inaccurate diagnosis and treatment plan, the processor is configured to generate a request for a consulting review of the diagnosis and treatment plan using the set of PA data.

In another aspect, a method is provided for determining that a diagnosis and treatment plan is inaccurate. The method is performed by a trained predictive server including a processor and a memory. The method includes receiving a set of prior authorization (PA) data associated with a medical claim for a patient, and determining that the set of PA data indicates that the medical claim is associated with a qualifying critical illness. The method further includes extracting component data from the set of PA data, and applying the extracted component data to a trained predictive model associated with the qualifying critical illness to determine whether the medical claim is associated with an inaccurate diagnosis and treatment plan. Upon determining that the medical claim is associated with an inaccurate diagnosis and treatment plan, a request for a consulting review of the diagnosis and treatment plan is generated using the set of PA data.

In yet another aspect, a trained predictive system is provided for determining that a diagnosis and treatment plan is inaccurate. The trained predictive system includes a first claim database server including a database processor and a database memory. The database memory includes a set of prior authorization (PA) data associated with a medical claim for a patient. The database processor is configured to determine that the set of PA data indicates that the medical claim is associated with a qualifying critical illness. The trained predictive system further includes a trained predictive server in communication with the first claim database server. The trained predictive server includes a processor and a memory. The processor is configured to receive the set of PA data associated with the medical claim for the patient from the first claim database server. The processor is further configured to extract component data from the set of PA data, and apply the extracted component data to a trained predictive model associated with the qualifying critical illness to determine whether the medical claim is associated with an inaccurate diagnosis and treatment plan. The database processor is further configured to receive an indication that the medical claim is associated with the inaccurate diagnosis and treatment plan from the trained predictive server, and generate a request for a consulting review of the diagnosis and treatment plan using the set of PA data.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is a functional block diagram of an example insurance claim processing system;

FIG. 2 is a functional block diagram of an example computing device;

FIG. 3 is a functional block diagram of an example trained predictive system that may be deployed within the system of FIG. 1 using the computing device shown in FIG. 2;

FIG. 4 is a flow diagram representing an example method for determining that a diagnosis and treatment plan is inaccurate from the perspective of the trained predictive server shown in FIG. 3;

FIG. 5 is a functional block diagram of an example system including a high-volume pharmacy;

FIG. 6 is a functional block diagram of an example pharmacy fulfillment device, which may be deployed within the system of FIG. 5;

FIG. 7 is a functional block diagram of an example order processing device, which may be deployed within the system of FIG. 5;

FIG. 8 is a block diagram of an example patient management platform that may be deployed within the system of FIG. 5, according to some examples; and

FIG. 9 is a functional block diagram of an example neural network that can be used for the inference engine or other functions (e.g., engines) as described herein to produce a predictive model.

In the drawings, reference numbers may be reused to identify similar and/or identical elements.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

As used herein, the term “feature selection” refers to the process of selecting a subset of relevant features (e.g., variables or predictors) that are used in the machine learning system to define data models. Feature selection may alternatively be described as variable selection, attribute selection, or variable subset selection. The feature selection process of the machine learning system described herein allows the machine learning system to simplify models to make them easier to interpret, reduce the time to train the systems, reduce overfitting, enhance generalization, and avoid problems in dynamic optimization. The data models described herein may include known data models and/or novel data models.

The machine learning systems and methods described herein are configured to address known technological problems confronting computing systems and networks that process data sets, specifically the lack of known static relationships between data sets and certain data characteristics.

In many examples, the reliability and accuracy of diagnostic and treatment determinations are important aspects of claim processing. In healthcare systems, healthcare providers typically provide (directly or indirectly) data associated with a patient upon making a diagnosis and determining a treatment plan. Typically, a set of relevant data (e.g., diagnosis, treatment plan) is provided in the context of a prior authorization (PA) request. PA requests are typically required by healthcare insurers after a physician prescribes such a treatment plan in order to confirm that the proposed treatment is covered by the insurer. Healthcare providers generate data related to treatment outcomes. Healthcare insurers also have access, directly or indirectly, to data related to treatment outcomes.

The consequences of an inaccurate diagnosis and/or treatment plan may dramatically affect the health and outcome of a patient, as well as incur significant financial and resource costs. Such consequences are elevated in the context of critical illnesses, and particularly in the context of chronic and terminal illnesses such as cancer. Moreover, while certain data relationships associated with the risk of misdiagnosis and/or mistreatment are known, assessing the reliability and accuracy of diagnoses and treatments associated with at least some illnesses is indeterminate or exacerbated because methodology for diagnoses and treatments of such illnesses is constantly evolving. As such, while static models may be applied using the methods and systems described herein, machine learning models are also contemplated and described herein. In this manner, the examples described herein are configured to systematically improve over time as illnesses are better understood and innovations, discoveries, and other developments in diagnoses and treatments continue to emerge.

The described machine learning systems and methods solve a technological problem related to unreliable data that cannot be otherwise resolved using known methods and technologies. In particular, the proposed approach of using machine learning to train a model to assess the diagnostic or treatment determinations is a significant technological improvement in the technological field of health and data sciences. By determining that a medical claim is associated with an inaccurate diagnosis or treatment plan as described herein, at least some medical claims can be systematically managed, enabling computing systems to reduce or mitigate the amount of storage space, bandwidth, processing power, etc. used on unreliable data (e.g., inaccurate diagnosis or treatment plan) and ultimately improve workflows, runtime performance, and data quality. Further, the proposed approach includes active re-training to ensure predictive accuracy and provide real-world benefits. For example, reducing the number of false negatives for illnesses may lead to earlier detection, reliable diagnoses, earlier treatment, appropriate treatment plans, and improved outcomes, and reducing the number of false positives may reduce the need for confirmatory testing and mitigate the risk of improper and/or unwarranted treatment. Moreover, increasing predictivity enables patients, healthcare providers, and insurers to rely on a diagnosis and treatment plan with greater confidence.

Generally, the systems and methods described apply a cyclical process of (a) defining a cohort for pattern identification that may be defined based on diagnosed illness types aggregating across at least one of: (i) patients, (ii) providers, and (iii) geographical regions; (b) identifying prior authorization (PA) or claim data associated with relevant critical claims within the cohort; (c) collecting or extracting data from PA data for the cohort; (d) determining patterns or relationships between PA data and outcomes for the cohort; (e) creating a data model based on the patterns or relationships; and (f) refining the data model with new data. The data model may be applied by the predictive server to PA data for incoming claims to identify likely misdiagnoses or mistreatment, and to recommend a clinical consultation (or second opinion) to reassess the diagnoses or treatment plan for such identified claims.

As described above, the systems and methods define cohorts based on diagnosed illness (or condition) type aggregating across a variety of groupings. In an example embodiment, the relevant illnesses or conditions that define the cohorts include at least one of breast cancer, non-small cell lung cancer, bone cancer, soft tissue sarcoma, colorectal cancer, central nervous system cancer, Hodgkin disease, and multiple myeloma. In the example embodiment, these conditions are selected because they are complex illnesses with a variety of possible underlying diagnoses and treatments. In other examples, the cohorts may include other chronic or terminal illnesses. It is contemplated that the systems and methods described may be relevant for a variety of such illnesses including autoimmune diseases and neurological diseases.

As described above, the machine learning models analyze the underlying PA and outcome data to determine certain data models that can be used to identify possible misdiagnoses or mistreatment based on composite PA data. Such models may include some of the following: (i) clinical indications with a high rate of misdiagnosis; (ii) clinical indications with a high rate of redirection (i.e., the diagnoses were not complete); (iii) healthcare providers associated with more frequent determinations of incorrect diagnoses or treatments; (iv) institutions associated with more frequent determinations of incorrect diagnoses or treatments; (vi) patient information including demographic data (e.g., age, gender, sex), relevant health biometric data, geographic data, and history data associated with frequent determinations of incorrect diagnoses or treatments. In many examples, the systems and methods described may create additional data models with combinations of such models. In some examples, the systems and methods may incorporate additional extrinsic data to create models including, for example, clinical research data, imaging data, and other health system data.

A trained predictive system for determining that a diagnosis and treatment plan is inaccurate is provided. The trained predictive system includes a first claim database server with a database processor and a database memory. The first claim database server includes a set of prior authorization (PA) data associated with a medical claim for a patient. In operation, the first claim database server receives requests from computing devices associated with healthcare providers including, for example, provider computing devices, hospital computing devices, and clinic computing devices. More specifically, when a provider makes a determination of a diagnosis and/or treatment plan for a particular patient, a PA request is typically submitted to an insurer associated with the first claim database. The PA request may include a variety of information related to the diagnosis and treatment of a particular patient. For example, PA requests may include some or all of: diagnostic data, treatment data, patient demographic data, patient geographic data, patient socioeconomic data, healthcare provider data, and healthcare provider reputation data. PA requests may also include relevant information for claim processing including insurer data, insurer identifiers, insured data, insured identifiers, and coverage data.

The trained predictive system also includes a trained predictive server that is in communication with the first claim database server. The trained predictive server includes a processor and a memory. The trained predictive server is configured to receive the set of prior authorization (PA) data associated with the medical claim for the patient from the first claim database. In some examples, the first claim database server and/or trained predictive server may be configured to determine whether the set of PA data indicates that the medical claim is associated with a qualifying critical illness. For example, the first claim database server and/or trained predictive server may identify or receive a list of qualifying critical illnesses from a storage device (e.g., a first data warehouse server) and apply the list to the set of PA data to determine whether the set of PA data indicates that the medical claim is associated with a qualifying critical illness, such as breast cancer, non-small cell lung cancer, bone cancer, soft tissue sarcoma, colorectal cancer, central nervous system cancer, Hodgkin disease, and multiple myeloma. The list of qualifying critical illnesses may include any illness or condition that allows or enables the trained predictive system to operate as described herein.

The trained predictive server is configured to extract component data from the set of PA data and apply the extracted component data to a trained predictive model to determine whether the medical claim is associated with an inaccurate diagnosis and treatment plan. In at least some examples, the trained predictive server identifies or receives a list of features for extraction, and extracts the component data, based on the list of features, from the set of PA data. The list of features may be predefined or derived. In some examples, the list of features are determined to include those features that are likely to indicate or influence an inaccurate diagnosis or treatment plan. The list of features may include, without limitation, diagnostic data, treatment data, patient demographic data, patient geographic data, patient socioeconomic data, healthcare provider data, and/or healthcare provider reputation data.

In some examples, the trained predictive server may quantify the extracted component data to determine a probability score or computation that describes or indicates a likelihood of whether the medical claim is associated with an inaccurate diagnosis and treatment plan. Probability scoring or computation may be valuable when the determination of inaccuracy is not obtainable in a binary fashion. The probability score may be compared against one or more predetermined thresholds to evaluate inaccuracies in the diagnosis and treatment plan and then determine an appropriate follow-up action. For example, a predetermined threshold may be set at 80% (or other suitable percentage) to distinguish medical claims that require a second opinion (e.g., because they are at least 80% likely to be associated with an inaccurate diagnosis and treatment plan) from medical claims that do not require a second opinion (e.g., because they are less than 80% likely to be associated with an inaccurate diagnosis and treatment plan). Additionally or alternatively, a predetermined threshold may be set at 21% (or other suitable percentage) to distinguish medical claims that are approved for processing (e.g., because they are less than 21% likely to be associated with an inaccurate diagnosis and treatment plan) from medical claims that are not (yet) approved for processing (e.g., because they are at least 21% likely to be associated with an inaccurate diagnosis and treatment plan). In some examples, the trained predictive server may be configured to generate or transmit an alert to request additional input (e.g., from a systems analyst, physician, or other technical or health care professional) regarding whether a second opinion is required and/or a medical claim is approved for processing when the medical claim is at least 21% (or other suitable percentage) and less than 80% (or other suitable percentage) likely to be associated with an inaccurate diagnosis and treatment plan. In operation, any suitable threshold (and any number of thresholds) may be applied depending on, e.g., the medical claim, the diagnosis and treatment plan, or other criteria being evaluated, and the comparisons between one or more thresholds and likelihoods may include statistical analyses (e.g., standard deviations), and may utilize rounding or other suitable approximations.

Upon determining that the medical claim is associated with an inaccurate diagnosis and treatment plan, the trained predictive server may generate a request for a consulting review of the diagnosis and treatment plan using the set of PA data. Additionally or alternatively, the request may be generated by the first claim database server. In any case, a consulting review provider may be identified based on the set of PA data, and the request may be transmitted to a computing device associated with the consulting review provider. In some examples, the first claim database server and/or trained predictive server may be configured to identify a list of candidate consulting review providers, wherein each candidate consulting review provider is associated with candidate provider reputation data and candidate provider location data, and to identify the consulting review provider from the list of candidate consulting review providers based on a comparison of the set of PA data to the candidate provider reputation data and the candidate provider location data.

A machine learning system is provided for training a predictive model to determine whether a diagnosis and treatment plan for a patient is inaccurate. For example, the machine learning system may apply a method to create and/or train the data models described herein. In some examples, the machine learning system includes the trained predictive server and a first data warehouse server in communication with the trained predictive server. The first data warehouse server may include a data warehouse processor and a data warehouse memory including data that may be used to create and/or train one or more data models. The data warehouse memory may include, for example, a plurality of historical prior authorization (PA) data and a plurality of result data associated with the historical PA data. Each historical PA data and associated result data is further associated with a patient claim. The result data may indicate whether each associated patient claim successfully processed.

A set of feature data may be extracted from each of the historical PA data. In at least some examples, the trained predictive server determines a list of features for extraction and extracts the feature data, based on the list of features, from the plurality of historical PA data. In some examples, the list of features are determined to include those features that are likely to indicate or influence an inaccurate diagnosis or treatment plan. Historical PA data that describes or indicates an inaccurate diagnosis or treatment plan may be associated, for example, with result data that describes or indicates an adverse outcome. The list of features may include, without limitation, diagnostic data, treatment data, patient demographic data, patient geographic data, patient socioeconomic data, healthcare provider data, and/or healthcare provider reputation data.

In some examples, the trained predictive server may be configured to apply the extracted feature data to a data model to determine whether the data model is reliable (e.g., whether the data model is configured to accurately determine whether a diagnosis and treatment plan associated with a medical claim is accurate or inaccurate). The trained predictive server may implement a feature selection process using one or more machine learning algorithms to facilitate improving a reliability of the data model. In addition to selecting one or more features, the machine learning algorithms may also determine one or more algorithms for use in selecting the features.

The trained predictive server may also be configured to extract a set of retraining feature data from each of the historical PA data and retrain one or more data models based on the retraining feature data. For example, the trained predictive server may identify or receive a first portion of the plurality of historical PA data and an associated first portion of the plurality of result data for use in creating and/or training one or more data models based on the first portion of the plurality of historical PA data and the first portion of the plurality of result data. The trained predictive server may then identify or receive a second portion of the plurality of historical PA data and an associated second portion of the plurality of result data for use in “retraining” one or more data models based on the second portion of the plurality of historical PA data and the second portion of the plurality of result data. In this manner, models can be iteratively or dynamically retrained as new historical PA data and associated result data is generated.

Generally, the systems and methods described herein are configured to perform at least the following steps: receiving a set of prior authorization (PA) data associated with a medical claim for a patient; identifying a list of qualifying critical illnesses from a storage device in communication with the trained predictive server; applying the list to the set of PA data to determine whether the set of PA data indicates that the medical claim is associated with a qualifying critical illness; determining that the set of PA data indicates that the medical claim is associated with a qualifying critical illness; identifying a list of features for extraction; extracting component data from the set of PA data; applying the extracted component data to the trained predictive model to determine whether the medical claim is associated with an inaccurate diagnosis and treatment plan; determining whether the medical claim is associated with an inaccurate diagnosis and treatment plan; determining a likelihood of whether the medical claim is associated with an inaccurate diagnosis and treatment plan; comparing the likelihood of whether the medical claim is associated with an inaccurate diagnosis and treatment plan against a predetermined threshold to evaluate inaccuracies in the diagnosis and treatment plan; identifying a list of candidate consulting review providers, wherein each candidate consulting review partner is associated with candidate provider reputation data and candidate provider location data; identifying a consulting review provider based on the set of PA data; identifying the consulting review provider from the list of candidate consulting review providers based on a comparison of the set of PA data to the candidate provider reputation data and the candidate provider location data; generating a request for a consulting review of the diagnosis and treatment plan using the set of PA data (e.g., on condition that the likelihood of whether the medical claim is associated with an inaccurate diagnosis and treatment plan is greater than an upper predetermined threshold); transmitting the request to a computing device associated with the consulting review provider; generating a request that the medical claim associated with the set of PA data be processed (e.g., on condition that the likelihood of whether the medical claim is associated with an inaccurate diagnosis and treatment plan is less than a lower predetermined threshold); generating or transmitting an alert to a secondary device to request input (e.g., on condition that the likelihood of whether the medical claim is associated with an inaccurate diagnosis and treatment plan is greater than a lower predetermined threshold and less than an upper predetermined threshold); determining a list of features for extraction; extracting feature data, based on the list of features, from a plurality of historical PA data; applying the extracted feature data to the data model to determine whether the data model is reliable; generating or modifying a data model based on the plurality of historical PA data and a plurality of result data associated with the plurality of historical PA data; receiving a first portion of the plurality of historical PA data and an associated first portion of the plurality of result data; and/or receiving a second portion of the plurality of historical PA data and an associated second portion of the plurality of result data.

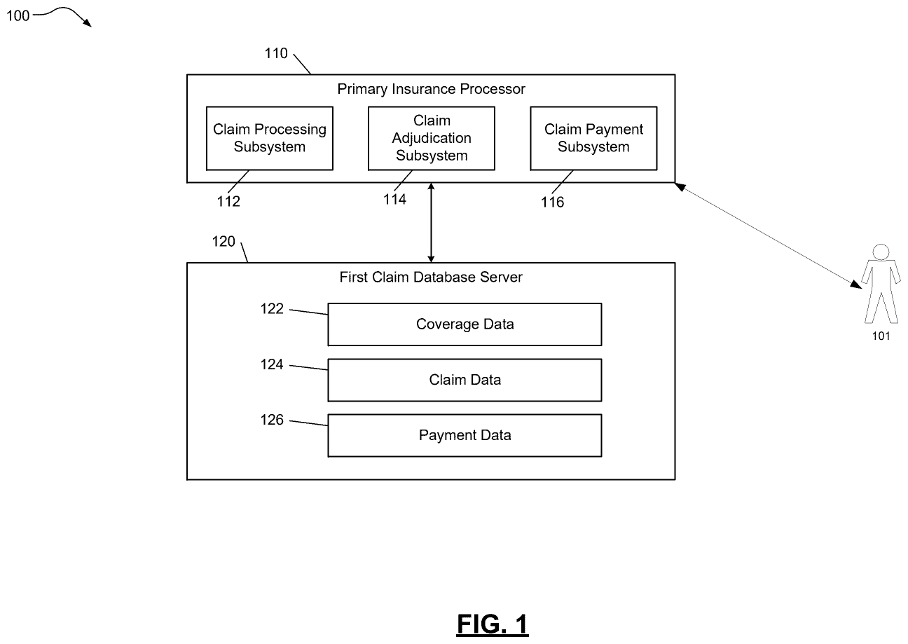

FIG. 1 is a functional block diagram of an example insurance claim processing system 100. Insurance processor system 110 includes subsystems 112, 114, and 116 capable of providing claim processing, claim adjudication, and claim payment respectively. First claim database server 120 includes necessary information on its underlying database. Specifically, first claim database server 120 includes coverage data 122, claim data 124, and payment data 126.

In operation, users such as user 101 may interact with insurance processor system 110. User 101 may be a healthcare provider, a patient, or any other suitable user involved in creating or reviewing claims. As described herein, in at least some examples, user 101 is a healthcare provider rendering diagnoses and/or treatment plans that are submitted in PA data to an insurer associated with insurance processor system 110.

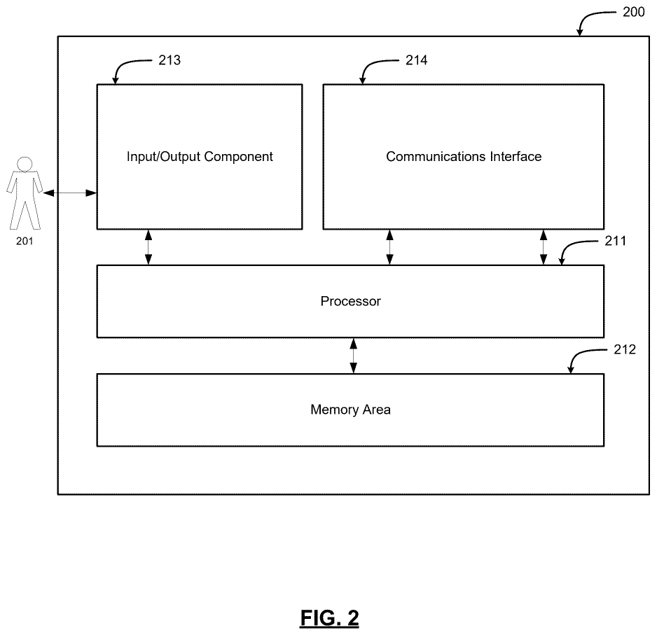

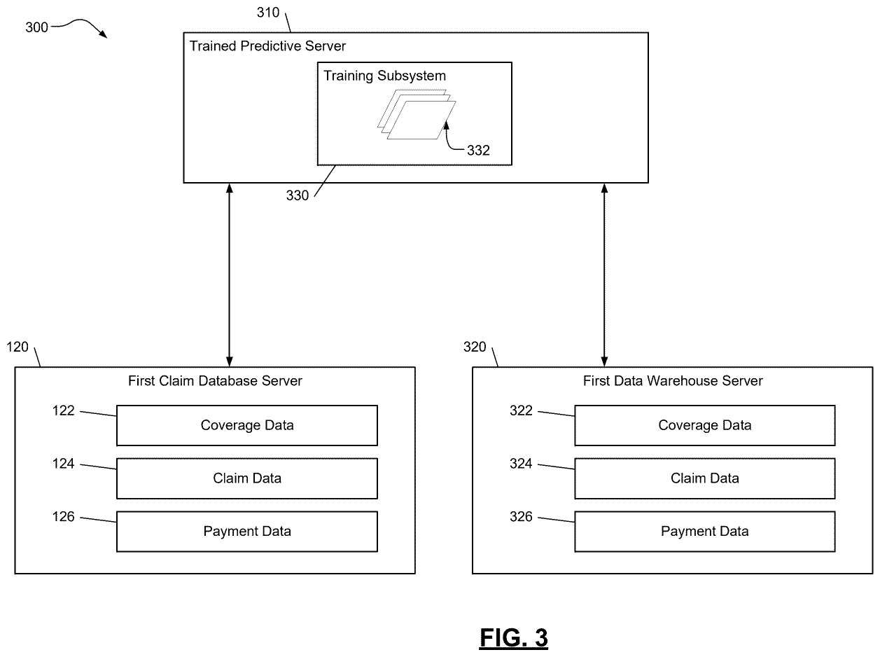

FIG. 2 is a functional block diagram of an example computing device that may be used in the trained predictive system described herein. Specifically, computing device 200 illustrates an example configuration of a computing device for the systems shown herein, and particularly in FIGS. 1 and 3. Computing device 200 illustrates an example configuration of a computing device operated by a user 201 in accordance with one embodiment of the present invention. Computing device 200 may include, but is not limited to, first claim database server, trained predictive server, first data warehouse server, and first predictive server, and other user systems, and other server systems. Computing device 200 may also include servers, desktops, laptops, mobile computing devices, stationary computing devices, computing peripheral devices, smart phones, wearable computing devices, medical computing devices, and vehicular computing devices. In some variations, computing device 200 may be any computing device capable of the described methods for predicting that PA data includes an incorrect diagnosis or treatment plan. In some variations, the characteristics of the described components may be more or less advanced, primitive, or non-functional.

In an example embodiment, computing device 200 includes a processor 211 for executing instructions. In some embodiments, executable instructions are stored in a memory area 212. Processor 211 may include one or more processing units, for example, a multi-core configuration. Memory area 212 is any device allowing information such as executable instructions and/or written works to be stored and retrieved. Memory area 212 may include one or more computer readable media.

Computing device 200 also includes at least one input/output component 213 for receiving information from and providing information to user 201 (e.g., user 101). In some examples, input/output component 213 may be of limited functionality or non-functional as in the case of some wearable computing devices. In other examples, input/output component 213 is any component capable of conveying information to or receiving information from user 201. In some embodiments, input/output component 213 includes an output adapter such as a video adapter and/or an audio adapter. Input/output component 213 may alternatively include an output device such as a display device, a liquid crystal display (LCD), organic light emitting diode (OLED) display, or “electronic ink” display, or an audio output device, a speaker or headphones. Input/output component 213 may also include any devices, modules, or structures for receiving input from user 201. Input/output component 213 may therefore include, for example, a keyboard, a pointing device, a mouse, a stylus, a touch sensitive panel, a touch pad, a touch screen, a gyroscope, an accelerometer, a position detector, or an audio input device. A single component such as a touch screen may function as both an output and input device of input/output component 213. Input/output component 213 may further include multiple sub-components for carrying out input and output functions.

Computing device 200 may also include a communications interface 214, which may be communicatively coupleable to a remote device such as a remote computing device, a remote server, or any other suitable system. Communication interface 214 may include, for example, a wired or wireless network adapter or a wireless data transceiver for use with a mobile phone network, Global System for Mobile communications (GSM), 3G, 4G, or other mobile data network or Worldwide Interoperability for Microwave Access (WIMAX). Communications interface 214 is configured to allow computing device 200 to interface with any other computing device or network using an appropriate wireless or wired communications protocol such as, without limitation, BLUETOOTH®, Ethernet, or IEEE 802.11. Communications interface 214 allows computing device 200 to communicate with any other computing devices with which it is in communication or connection.

FIG. 3 is a functional block diagram of a trained predictive system 300 that may be deployed within system 100 (shown in FIG. 1) using the computing device 200 (shown in FIG. 2). Specifically, trained predictive system 300 includes a trained predictive server 310 which is in communication with at least first claim database server 120, which may be associated with a first healthcare provider. For example, once the first healthcare provider makes a determination of a diagnosis and/or treatment plan for a particular patient, the first claim database server 120 may include PA data associated with the patient (e.g., diagnostic data, treatment data, patient demographic data, patient geographic data, patient socioeconomic data) and/or the first healthcare provider (e.g., healthcare provider data, healthcare provider reputation data). The trained predictive server 310 is also in communication with a first data warehouse sevrer 320, which includes a plurality of historical prior authorization (PA) data associated with a plurality of patients and/or healthcare providers as well as a plurality of result data associated with the historical PA data. For example, as shown in FIG. 3, the first data warehouse server 320 may include coverage data 322, claim data 324, and payment data 326. In this manner, coverage data 122 and 322 and claim data 124 and 324 may include the relevant PA data and outcome data described herein.

Trained predictive server 310 includes subsystems capable of performing the methods described herein and, more specifically, training subsystem 330 generates and/or defines one or more models 332 for predicting misdiagnoses and mistreatment. Models 332 may be generated using data in first data warehouse server 320 (e.g., coverage data 322, claim data 324). Trained predictive server 310 is configured to apply such models 332 to data in first claim database server 120 and, more specifically, to PA data included therein (e.g., coverage data 122, claim data 124) for determining whether a diagnosis and/or treatment plan is accurate.

Models 332 may be generated and/or modified to improve the reliability and accuracy of diagnostic and treatment determinations. For example, the trained predictive server 310 may create and/or refine one or more models 332 by applying a cyclical process of defining a cohort; collecting or extracting historical PA data for the cohort, as well as the corresponding result data; determining patterns or relationships between the historical PA data and the corresponding result data, and selecting one or more features (e.g., variables or predictors) that have a correlation with inaccurate diagnoses and treatment plans. Such features are selected based on their propensity to indicate or influence an inaccurate diagnosis or treatment plan. For example, the trained predictive server 310 may conduct a correlation analysis between the historical PA data and result data and select one or more features based on a correlation coefficient between such features and result data describing or indicating an adverse outcome. Additionally or alternatively, one or more features may be selected based on the availability of testing and diagnostic tools. One or more machine learning algorithms may be used to select one or more features, as well as to select one or more machine learning algorithms for selecting the features. Example features may include, without limitation, diagnostic data, treatment data, patient demographic data, patient geographic data, patient socioeconomic data, healthcare provider data, and/or healthcare provider reputation data. In some examples, the cohort may be defined based on one or more illnesses including, without limitation, breast cancer, non-small cell lung cancer, bone cancer, soft tissue sarcoma, colorectal cancer, central nervous system cancer, Hodgkin disease, and/or multiple myeloma.

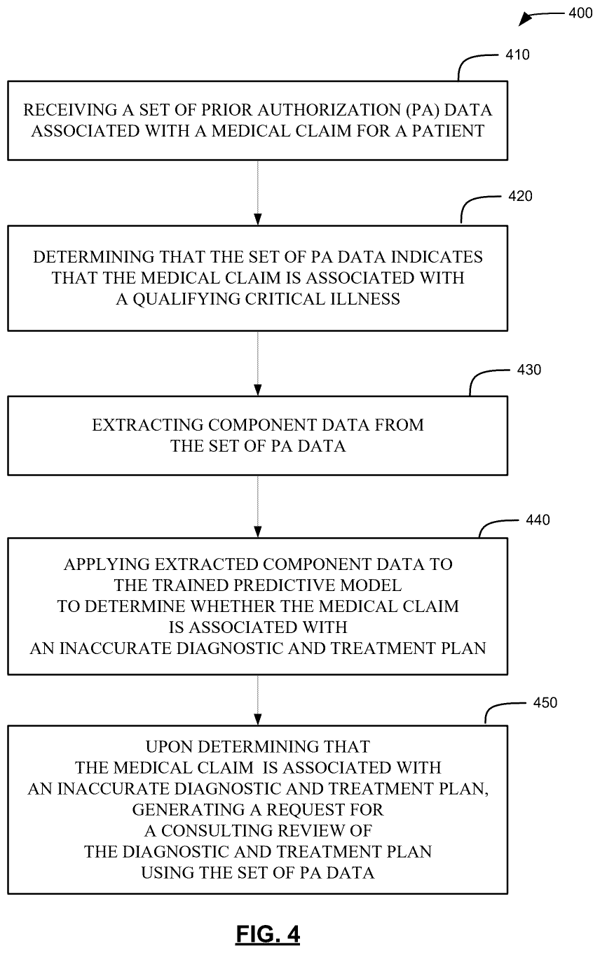

FIG. 4 is a flow diagram 400 representing a method for determining that a diagnosis and treatment plan is inaccurate from the perspective of the trained predictive server 310 (shown in FIG. 3). Specifically, trained predictive server 310 is configured to receive 410 a set of prior authorization (PA) data associated with a medical claim for a patient (e.g., claim data 124 or 324). For example, the PA data may include a diagnosis and treatment plan. In some examples, the trained predictive server 310 communicates with the first claim database server 120 to retrieve or collect other data associated with the medical claim and/or diagnosis and treatment plan (e.g., medical records, imaging, laboratory results, etc.).

Trained predictive server 310 may be configured to determine 420 whether the set of PA data indicates that the medical claim is associated with a qualifying critical illness (e.g., using coverage data 122 or 322). Trained predictive server 310 is further configured to extract 430 component data from the set of PA data. The features associated with the component data correspond to the features associated with the model 332. Example features may include, without limitation, diagnostic data, treatment data, patient demographic data, patient geographic data, patient socioeconomic data, healthcare provider data, and/or healthcare provider reputation data.

Trained predictive server 310 may be configured to apply 440 the extracted component data to the trained predictive model to determine whether the medical claim is associated with an inaccurate diagnosis and treatment plan. Based on the determination, the trained predictive server 310 may then determine an appropriate follow-up action. For example, if the medical claim is associated with an inaccurate diagnosis and treatment plan, the trained predictive server 310 may generate 450 a request for a consulting review of the diagnosis and treatment plan using the set of PA data and a flag that places a hold on the use of medical devices, e.g., automated pharmacy, chemotherapy machines, lasers, surgical (automated surgical equipment), and the like. If the model does not identify the medical diagnosis as a critical illness or a misdiagnosis of a critical illness, then the medical record is flagged as allowing the use of medical devices. This enables the inaccurate diagnosis and treatment plan to be modified or updated with an accurate diagnosis and treatment plan. In some examples, the trained predictive server 310 communicates with the first claim database server 120 and/or first data warehouse server 320 to retrieve or collect other data associated with the medical claim and/or diagnosis and treatment plan (e.g., medical records, imaging, laboratory results, etc.) and transmits such data to a computing device associated with the consulting review provider along with the request for the consulting review or upon receiving an affirmative response to the request.

In some examples, the trained predictive server 310 maybe configured to identify or select a first consulting review provider from a list of candidate consulting review providers. The first consulting review provider may be selected based on candidate provider reputation data and candidate provider location data, for example. For example, the first consulting review provider may be geographically diverse (e.g., candidate provider location data is different from patient and/or healthcare provider).

To facilitate determining whether the medical claim is associated with an inaccurate diagnosis and treatment plan, the trained predictive server 310 may quantify the extracted component data by determining a likelihood of whether the medical claim is associated with an inaccurate diagnosis and treatment plan. In some examples, the trained predictive server 310 may determine that the medical claim is associated with an inaccurate diagnosis and treatment plan if the likelihood satisfies a predetermined threshold (e.g., the likelihood is greater than or equal to 80% or other suitable percentage). On the other hand, the trained predictive server 310 may determine that the medical claim is not associated with an inaccurate diagnosis and treatment plan (or is associated with an accurate diagnosis and treatment plan) when the likelihood does not satisfy a predetermined threshold (e.g., the likelihood is less than or equal to 20% or other suitable percentage). In some examples, the trained predictive server 310 may generate or transmit an alert to request additional input (e.g., from a systems analyst, physician, or other technical or health care professional) regarding whether the medical claim is associated with an inaccurate diagnosis and treatment plan when the likelihood is between a lower predetermined threshold (e.g., 20% or other suitable percentage) and an upper predetermined threshold (80% or other suitable percentage).

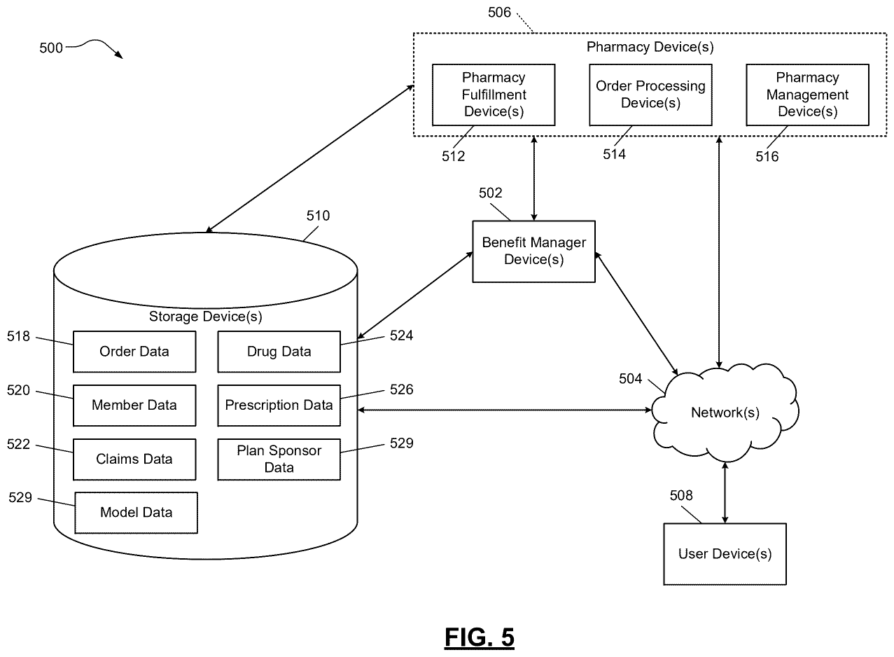

FIG. 5 is a block diagram of an example implementation of a system 500 for a high-volume pharmacy, which may receive control signals from the system running the model to not fill a prescription or fill a prescription. While the system 500 is generally described as being deployed in a high-volume pharmacy or a fulfillment center (for example, a mail order pharmacy, a direct delivery pharmacy, etc.), the system 500 and/or components of the system 500 may otherwise be deployed (for example, in a lower-volume pharmacy, a specialty pharmacy, etc.). A high-volume pharmacy may be a pharmacy that is capable of filling at least some prescriptions mechanically. The system 500 may include a benefit manager device 502 and a pharmacy device a06 in communication with each other directly and/or over a network 504.

The system 500 may also include one or more user device(s) 508. A user, such as a pharmacist, patient, data analyst, health plan administrator, etc., may access the benefit manager device 502 or the pharmacy device 506 using the user device 508. The user device 508 may be a desktop computer, a laptop computer, a tablet, a smartphone, etc.

The benefit manager device 502 is a device operated by an entity that is at least partially responsible for creation and/or management of the pharmacy or drug benefit. While the entity operating the benefit manager device 502 is typically a pharmacy benefit manager (PBM), other entities may operate the benefit manager device 502 on behalf of themselves or other entities (such as PBMs). For example, the benefit manager device 502 may be operated by a health plan, a retail pharmacy chain, a drug wholesaler, a data analytics or other type of software-related company, etc. In some implementations, a PBM that provides the pharmacy benefit may provide one or more additional benefits, including a medical or health benefit, a dental benefit, a vision benefit, a wellness benefit, a radiology benefit, a pet care benefit, an insurance benefit, a long-term care benefit, a nursing home benefit, etc. The PBM may, in addition to its PBM operations, operate one or more pharmacies. The pharmacies may be retail pharmacies, mail order pharmacies, etc. In an example embodiment, the benefit manager device 502 includes the described machine learning systems and methods, e.g., the insurance claim processing system 100 with the insurance processor system 110 and the first claim database server 120, the trained predictive server 310, and the first data warehouse server 320. In other examples, the first data warehouse server 320 and the first claim database server 120 can be in the storage devices 510.

Some of the operations of the PBM that operates the benefit manager device 502 may include the following activities and processes. A member (or a person on behalf of the member) of a pharmacy benefit plan may obtain a medical diagnosis from a medical provider or a prescription drug at a retail pharmacy location (e.g., a location of a physical store) from a pharmacist or a pharmacist technician. The member may also obtain the prescription drug through mail order drug delivery from a mail order pharmacy location, such as the system 500. In some implementations, the member may obtain the prescription drug directly or indirectly through the use of a machine, such as a kiosk, a vending unit, a mobile electronic device, or a different type of mechanical device, electrical device, electronic communication device, and/or computing device. Such a machine may be filled with the prescription drug in prescription packaging, which may include multiple prescription components, by the system 500. The pharmacy benefit plan is administered by or through the benefit manager device 502. The medical diagnosis and/or the prescription is sent to the trained predictive model 332 that is part of the trained predictive server 310.

The member may have a copayment for the prescription drug that reflects an amount of money that the member is responsible to pay the pharmacy for the prescription drug. The money paid by the member to the pharmacy may come from, as examples, personal funds of the member, a health savings account (HSA) of the member or the member's family, a health reimbursement arrangement (HRA) of the member or the member's family, or a flexible spending account (FSA) of the member or the member's family. In some instances, an employer of the member may directly or indirectly fund or reimburse the member for the copayments. In an example embodiment, the co-pay is calculated separate from or after operation of the trained predictive model 332.

The amount of the copayment required by the member may vary across different pharmacy benefit plans having different plan sponsors or clients and/or for different prescription drugs. The member's copayment may be a flat copayment (in one example, $10), coinsurance (in one example, 10%), and/or a deductible (for example, responsibility for the first $ 500 of annual prescription drug expense, etc.) for certain prescription drugs, certain types and/or classes of prescription drugs, and/or all prescription drugs. The copayment may be stored in a storage device 510 or determined by the benefit manager device 502.

In some instances, the member may not pay the copayment or may only pay a portion of the copayment for the prescription drug. For example, if a usual and customary cost for a generic version of a prescription drug is $4, and the member's flat copayment is $20 for the prescription drug, the member may only need to pay $4 to receive the prescription drug. In another example involving a worker's compensation claim, no copayment may be due by the member for the prescription drug.

In addition, copayments may also vary based on different delivery channels for the prescription drug. For example, the copayment for receiving the prescription drug from a mail order pharmacy location may be less than the copayment for receiving the prescription drug from a retail pharmacy location.

In conjunction with receiving a copayment (if any) from the member and dispensing the prescription drug to the member, the pharmacy submits a claim to the PBM for the prescription drug. After receiving the claim, the PBM (such as by using the benefit manager device 502) may perform certain adjudication operations including verifying eligibility for the member, identifying/reviewing an applicable formulary for the member to determine any appropriate copayment, coinsurance, and deductible for the prescription drug, and performing a drug utilization review (DUR) for the member. Further, the PBM may provide a response to the pharmacy, the model (for example, the system 100 or the system 500) following performance of at least some of the aforementioned operations.

As part of the adjudication, a plan sponsor (or the PBM on behalf of the plan sponsor) ultimately reimburses the pharmacy for filling the prescription drug when the prescription drug was successfully adjudicated. The aforementioned adjudication operations generally occur before the copayment is received and the prescription drug is dispensed. However in some instances, these operations may occur simultaneously, substantially simultaneously, or in a different order. In addition, more or fewer adjudication operations may be performed as at least part of the adjudication process. In an example, the adjudication occurs after the processing through the trained model 332 in the trained predictive server 310

The amount of reimbursement paid to the pharmacy by a plan sponsor and/or money paid by the member may be determined at least partially based on types of pharmacy networks in which the pharmacy is included. In some implementations, the amount may also be determined based on other factors. For example, if the member pays the pharmacy for the prescription drug without using the prescription or drug benefit provided by the PBM, the amount of money paid by the member may be higher than when the member uses the prescription or drug benefit. In some implementations, the amount of money received by the pharmacy for dispensing the prescription drug and for the prescription drug itself may be higher than when the member uses the prescription or drug benefit. Some or all of the foregoing operations may be performed by executing instructions stored in the benefit manager device 502 and/or an additional device, e.g., the trained predictive server 310.

Examples of the network 504 include a Global System for Mobile Communications (GSM) network, a code division multiple access (CDMA) network, 3rd Generation Partnership Project (3GPP), an Internet Protocol (IP) network, a Wireless Application Protocol (WAP) network, or an IEEE 802.11 standards network, as well as various combinations of the above networks. The network 504 may include an optical network. The network 504 may be a local area network or a global communication network, such as the Internet. In some implementations, the network 504 may include a network dedicated to prescription orders: a prescribing network, such as the electronic prescribing network operated by Surescripts of Arlington, Virginia.

Moreover, although the system shows a single network 504, multiple networks can be used. The multiple networks may communicate in series and/or in parallel with each other to link the devices 502-510.

The pharmacy device 506 may be a device associated with a retail pharmacy location (e.g., an exclusive pharmacy location, a grocery store with a retail pharmacy, or a general sales store with a retail pharmacy) or other type of pharmacy location at which a member attempts to obtain a prescription. The pharmacy may use the pharmacy device 506 to submit the claim to the PBM for adjudication.

Additionally, in some implementations, the pharmacy device 506 may enable information exchange between the pharmacy and the PBM. For example, this may allow the sharing of member information such as drug history that may allow the pharmacy to better service a member (for example, by providing more informed therapy consultation and drug interaction information). In some implementations, the benefit manager device 102 may track prescription drug fulfillment and/or other information for users that are not members, or have not identified themselves as members, at the time (or in conjunction with the time) at which they seek to have a prescription filled at a pharmacy.

The pharmacy device 506 may include a pharmacy fulfillment device 512, an order processing device 514, and a pharmacy management device 516 in communication with each other directly and/or over the network 504. The order processing device 514 may receive information regarding filling prescriptions and may direct an order component to one or more devices of the pharmacy fulfillment device 512 at a pharmacy. In an example, pharmacy device 506 may receive a no model review signal indicating that the prescription is not a critical illness that requires review by the trained predictive model and can proceed with processing the prescription for fulfillment. In an example, pharmacy device 506 may receive a pause signal hold processing the prescription order as model review is required. In an example and after trained model 332 review, pharmacy device 506 may receive a model review complete signal that indicates wither the pharmacy device 506 cannot process the prescription or may process the prescription. The pharmacy fulfillment device 512 may fulfill, dispense, aggregate, and/or pack the order components of the prescription drugs in accordance with one or more prescription orders directed by the order processing device 514.

In general, the order processing device 514 is a device located within or otherwise associated with the pharmacy to enable the pharmacy fulfillment device 512 to fulfill a prescription and dispense prescription drugs. In some implementations, the order processing device 514 may be an external order processing device separate from the pharmacy and in communication with other devices located within the pharmacy.

For example, the external order processing device may communicate with an internal pharmacy order processing device and/or other devices located within the system 500. In some implementations, the external order processing device may have limited functionality (e.g., as operated by a user requesting fulfillment of a prescription drug), while the internal pharmacy order processing device may have greater functionality (e.g., as operated by a pharmacist).

The order processing device 514 may track the prescription order as it is fulfilled by the pharmacy fulfillment device 512. The prescription order may include one or more prescription drugs to be filled by the pharmacy. The order processing device 514 may make pharmacy routing decisions and/or order consolidation decisions for the particular prescription order. The pharmacy routing decisions include what device(s) in the pharmacy are responsible for filling or otherwise handling certain portions of the prescription order. The order consolidation decisions include whether portions of one prescription order or multiple prescription orders should be shipped together for a user or a user's family. The order processing device 514 may also track and/or schedule literature or paperwork associated with each prescription order or multiple prescription orders that are being shipped together. In an example, the results of the trained model 332 review are sent to the order processing device for inclusion with the literature or paperwork associated with the prescription order. In some implementations, the order processing device 514 may operate in combination with the pharmacy management device 516.

The order processing device 514 may include circuitry, a processor, a memory to store data and instructions, and communication functionality. The order processing device 514 is dedicated to performing processes, methods, and/or instructions described in this application. Other types of electronic devices may also be used that are specifically configured to implement the processes, methods, and/or instructions described in further detail below.

In some implementations, at least some functionality of the order processing device 514 may be included in the pharmacy management device 516. The order processing device 514 may be in a client-server relationship with the pharmacy management device 516, in a peer-to-peer relationship with the pharmacy management device 516, or in a different type of relationship with the pharmacy management device 516. The order processing device 514 and/or the pharmacy management device 516 may communicate directly (for example, such as by using a local storage) and/or through the network 504 (such as by using a cloud storage configuration, software as a service, etc.) with the storage device 510. In an example, the pharmacy management device 516 stores and operates a current version of the trained predictive model 332 with the training of the model being performed remote from the pharmacy management device 516.

The storage device 510 may include: non-transitory storage (for example, memory, hard disk, CD-ROM, etc.) in communication with the benefit manager device 502 and/or the pharmacy device 506 directly and/or over the network 504. The non-transitory storage may store order data 518, member data 520, claims data 522, drug data 524, prescription data 526, and/or plan sponsor data 528, as well as model data 529. Further, the system 500 may include additional devices, which may communicate with each other directly or over the network 504.

The order data 518 may be related to a prescription order. The order data may include the type of the prescription drug (for example, drug name and strength) and the quantity of the prescription drug. The order data 518 may also include data used for completion of the prescription, such as prescription materials. In general, prescription materials include an electronic copy of information regarding the prescription drug for inclusion with or otherwise in conjunction with the fulfilled prescription. The prescription materials may include electronic information regarding drug interaction warnings, recommended usage, possible side effects, expiration date, date of prescribing, supporting data for the results from the trained predictive model 332, etc. The order data 518 may be used by a high-volume fulfillment center to fulfill a pharmacy order.

In some implementations, the order data 518 includes verification information associated with fulfillment of the prescription in the pharmacy. For example, the order data 518 may include videos and/or images taken of (i) the prescription drug prior to dispensing, during dispensing, and/or after dispensing, (ii) the prescription container (for example, a prescription container and sealing lid, prescription packaging, etc.) used to contain the prescription drug prior to dispensing, during dispensing, and/or after dispensing, (iii) the packaging and/or packaging materials used to ship or otherwise deliver the prescription drug prior to dispensing, during dispensing, and/or after dispensing, and/or (iv) the fulfillment process within the pharmacy. Other types of verification information such as barcode data read from pallets, bins, trays, or carts used to transport prescriptions within the pharmacy may also be stored as order data 518.

The member data 520 includes information regarding the members associated with the PBM. The information stored as member data 520 may include personal information, personal health information, protected health information, etc. Examples of the member data 120 include name, age, date of birth, address (including city, state, and zip code), telephone number, e-mail address, medical history, prescription drug history, etc. In various implementations, the prescription drug history may include a prior authorization claim history—including the total number of prior authorization claims, approved prior authorization claims, and denied prior authorization claims, either by the trained predictive model 332 of by other processors. In various implementations, the prescription drug history may include previously filled claims for the member, including a date of each filled claim, a dosage of each filled claim, the drug type for each filled claim, a prescriber associated with each filled claim, and whether the drug associated with each claim is on a formulary (e.g., a list of covered medications).

In various implementations, the medical history may include whether and/or how well each member adhered to one or more specific therapies. The member data 520 may also include a plan sponsor identifier that identifies the plan sponsor associated with the member and/or a member identifier that identifies the member to the plan sponsor. The member data 520 may include a member identifier that identifies the plan sponsor associated with the user and/or a user identifier that identifies the user to the plan sponsor. In various implementations, the member data 520 may include an eligibility period for each member. For example, the eligibility period may include how long each member is eligible for coverage under the sponsored plan. The member data 520 may also include dispensation preferences such as type of label, type of cap, message preferences, language preferences, etc.

The member data 520 may be accessed by various devices in the pharmacy (for example, the high-volume fulfillment center, etc.) to obtain information used for fulfillment and shipping of prescription orders. In some implementations, an external order processing device operated by or on behalf of a member may have access to at least a portion of the member data 520 for review, verification, or other purposes.

In some implementations, the member data 520 may include information for persons who are users of the pharmacy but are not members in the pharmacy benefit plan being provided by the PBM. For example, these users may obtain drugs directly from the pharmacy, through a private label service offered by the pharmacy, the high-volume fulfillment center, or otherwise. In general, the terms “member” and “user” may be used interchangeably.

The claims data 522 includes information regarding pharmacy claims adjudicated by the PBM under a drug benefit program provided by the PBM for one or more plan sponsors. In general, the claims data 522 includes an identification of the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copayment/coinsurance amount, rebate information, and/or member eligibility, etc. Additional information may be included.

In some implementations, other types of claims beyond prescription drug claims may be stored in the claims data 522. For example, medical claims, dental claims, wellness claims, or other types of health-care-related claims for members may be stored as a portion of the claims data 522.

In some implementations, the claims data 522 includes claims that identify the members with whom the claims are associated. Additionally or alternatively, the claims data 522 may include claims that have been de-identified (that is, associated with a unique identifier but not with a particular, identifiable member). In various implementations, the claims data 522 may include a percentage of prior authorization cases for each prescriber that have been denied, and a percentage of prior authorization cases for each prescriber that have been approved.

The drug data 524 may include drug name (e.g., technical name and/or common name), other names by which the drug is known, active ingredients, an image of the drug (such as in pill form), etc. The drug data 524 may include information associated with a single medication or multiple medications. For example, the drug data 524 may include a numerical identifier for each drug, such as the U.S. Food and Drug Administration's (FDA) National Drug Code (NDC) for each drug.

The prescription data 526 may include information regarding prescriptions that may be issued by prescribers on behalf of users, who may be members of the pharmacy benefit plan—for example, to be filled by a pharmacy. Examples of the prescription data 526 include user names, medication or treatment (such as lab tests), dosing information, etc. The prescriptions may include electronic prescriptions or paper prescriptions that have been scanned. In some implementations, the dosing information reflects a frequency of use (e.g., once a day, twice a day, before each meal, etc.) and a duration of use (e.g., a few days, a week, a few weeks, a month, etc.).

In some implementations, the order data 518 may be linked to associated member data 520, claims data 522, drug data 524, and/or prescription data 526.

The plan sponsor data 528 includes information regarding the plan sponsors of the PBM. Examples of the plan sponsor data 528 include company name, company address, contact name, contact telephone number, contact e-mail address, etc.

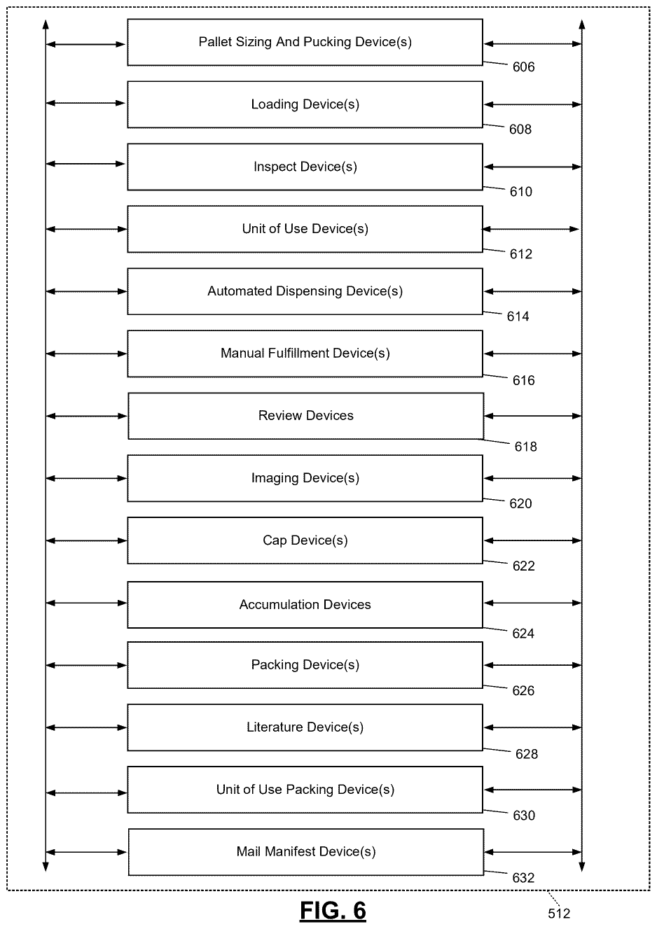

FIG. 6 illustrates the pharmacy fulfillment device 512 according to an example implementation. The pharmacy fulfillment device 512 may be used to process and fulfill prescriptions and prescription orders. After fulfillment, the fulfilled prescriptions are packed for shipping.

The pharmacy fulfillment device 512 may include devices in communication with the benefit manager device 502, the order processing device 514, and/or the storage device 510, directly or over the network 504. Specifically, the pharmacy fulfillment device 512 may include pallet sizing and pucking device(s) 606, loading device(s) 608, inspect device(s) 610, unit of use device(s) 612, automated dispensing device(s) 614, manual fulfillment device(s) 616, review devices 618, imaging device(s) 620, cap device(s) 622, accumulation devices 624, packing device(s) 626, literature device(s) 628, unit of use packing device(s) 630, and mail manifest device(s) 632. These devices may not operate to fulfill a prescription order until the trained predictive model 332 approves an individual order for fulfillment or the diagnosis associated with the prescription does not require review by the trained predictive model 332. Further, the pharmacy fulfillment device 512 may include additional devices, which may communicate with each other directly or over the network 504.

In some implementations, operations performed by one of these devices 606-632 may be performed sequentially, or in parallel with the operations of another device as may be coordinated by the order processing device 514. In some implementations, the order processing device 514 tracks a prescription with the pharmacy based on operations performed by one or more of the devices 606-632.

In some implementations, the pharmacy fulfillment device 512 may transport prescription drug containers, for example, among the devices 606-632 in the high-volume fulfillment center, by use of pallets. The pallet sizing and pucking device 606 may configure pucks in a pallet. A pallet may be a transport structure for a number of prescription containers, and may include a number of cavities. A puck may be placed in one or more than one of the cavities in a pallet by the pallet sizing and pucking device 606. The puck may include a receptacle sized and shaped to receive a prescription container. Such containers may be supported by the pucks during carriage in the pallet. Different pucks may have differently sized and shaped receptacles to accommodate containers of differing sizes, as may be appropriate for different prescriptions.

The arrangement of pucks in a pallet may be determined by the order processing device 514 based on prescriptions that the order processing device 514 decides to launch. The arrangement logic may be implemented directly in the pallet sizing and pucking device 606. Once a prescription is set to be launched, a puck suitable for the appropriate size of container for that prescription may be positioned in a pallet by a robotic arm or pickers. The pallet sizing and pucking device 606 may launch a pallet once pucks have been configured in the pallet.

The loading device 608 may load prescription containers into the pucks on a pallet by a robotic arm, a pick and place mechanism (also referred to as pickers), etc. In various implementations, the loading device 608 has robotic arms or pickers to grasp a prescription container and move it to and from a pallet or a puck. The loading device 608 may also print a label that is appropriate for a container that is to be loaded onto the pallet, and apply the label to the container. The pallet may be located on a conveyor assembly during these operations (e.g., at the high-volume fulfillment center, etc.).

The inspect device 610 may verify that containers in a pallet are correctly labeled and in the correct spot on the pallet. The inspect device 610 may scan the label on one or more containers on the pallet. Labels of containers may be scanned or imaged in full or in part by the inspect device 610. Such imaging may occur after the container has been lifted out of its puck by a robotic arm, picker, etc., or may be otherwise scanned or imaged while retained in the puck. In some implementations, images and/or video captured by the inspect device 610 may be stored in the storage device 510 as order data 518.

The unit of use device 612 may temporarily store, monitor, label, and/or dispense unit of use products. In general, unit of use products are prescription drug products that may be delivered to a user or member without being repackaged at the pharmacy. These products may include pills in a container (e.g., a box), pills in a blister pack, inhalers, etc. Prescription drug products dispensed by the unit of use device 612 may be packaged individually or collectively for shipping, or may be shipped in combination with other prescription drugs dispensed by other devices in the high-volume fulfillment center.

At least some of the operations of the devices 606-632 may be directed by the order processing device 514. For example, the manual fulfillment device 616, the review device 618, the automated dispensing device 614, and/or the packing device 626, etc. may receive instructions provided by the order processing device 514.

The automated dispensing device 614 may include one or more devices that dispense prescription drugs or pharmaceuticals into prescription containers in accordance with one or multiple prescription orders. In general, the automated dispensing device 614 may include mechanical and electronic components with, in some implementations, software and/or logic to facilitate pharmaceutical dispensing that would otherwise be performed in a manual fashion by a pharmacist and/or pharmacist technician. For example, the automated dispensing device 614 may include high-volume fillers that fill a number of prescription drug types at a rapid rate and blister pack machines that dispense and pack drugs into a blister pack. In an example, the blister packs can be generated by the structures and methods described in U.S. Pat. No. 11,779,518, granted 10 Oct. 2023 and U.S. Pat. No. 10,331,858, granted 25 Jun. 2019, which are both hereby incorporated by reference. Prescription drugs dispensed by the automated dispensing devices 614 may be packaged individually or collectively for shipping, or may be shipped in combination with other prescription drugs dispensed by other devices in the high-volume fulfillment center.

The manual fulfillment device 616 controls how prescriptions are manually fulfilled. For example, the manual fulfillment device 616 may receive or obtain a container and enable fulfillment of the container by a pharmacist or pharmacy technician. In some implementations, the manual fulfillment device 616 provides the filled container to another device in the pharmacy fulfillment devices 512 to be joined with other containers in a prescription order for a user or member.

In general, manual fulfillment may include operations at least partially performed by a pharmacist or a pharmacy technician. For example, a person may retrieve a supply of the prescribed drug, may make an observation, may count out a prescribed quantity of drugs and place them into a prescription container, etc. Some portions of the manual fulfillment process may be automated by use of a machine. For example, counting of capsules, tablets, or pills may be at least partially automated (such as through use of a pill counter). Prescription drugs dispensed by the manual fulfillment device 616 may be packaged individually or collectively for shipping, or may be shipped in combination with other prescription drugs dispensed by other devices in the high-volume fulfillment center.

The review device 618 may process prescription containers to be reviewed by a pharmacist for proper pill count, exception handling, prescription verification, etc. Fulfilled prescriptions may be manually reviewed and/or verified by a pharmacist, as may be required by state or local law. A pharmacist or other licensed pharmacy person who may dispense certain drugs in compliance with local and/or other laws may operate the review device 618 and visually inspect a prescription container that has been filled with a prescription drug. The pharmacist may review, verify, and/or evaluate drug quantity, drug strength, and/or drug interaction concerns, or otherwise perform pharmacist services. The pharmacist may also handle containers which have been flagged as an exception, such as containers with unreadable labels, containers for which the associated prescription order has been canceled, containers with defects, etc. In an example, the manual review can be performed at a manual review station.

The imaging device 620 may image containers once they have been filled with pharmaceuticals. The imaging device 620 may measure a fill height of the pharmaceuticals in the container based on the obtained image to determine if the container is filled to the correct height given the type of pharmaceutical and the number of pills in the prescription. Images of the pills in the container may also be obtained to detect the size of the pills themselves and markings thereon. The images may be transmitted to the order processing device 514 and/or stored in the storage device 510 as part of the order data 518.

The cap device 622 may be used to cap or otherwise seal a prescription container. In some implementations, the cap device 622 may secure a prescription container with a type of cap in accordance with a user preference (e.g., a preference regarding child resistance, etc.), a plan sponsor preference, a prescriber preference, etc. The cap device 622 may also etch a message into the cap, although this process may be performed by a subsequent device in the high-volume fulfillment center.

The accumulation device 624 accumulates various containers of prescription drugs in a prescription order. The accumulation device 624 may accumulate prescription containers from various devices or areas of the pharmacy. For example, the accumulation device 624 may accumulate prescription containers from the unit of use device 612, the automated dispensing device 614, the manual fulfillment device 616, and the review device 618. The accumulation device 624 may be used to group the prescription containers prior to shipment to the member.

The literature device 628 prints, or otherwise generates, literature to include with each prescription drug order. The literature may be printed on multiple sheets of substrates, such as paper, coated paper, printable polymers, or combinations of the above substrates. The literature printed by the literature device 628 may include information required to accompany the prescription drugs included in a prescription order, other information related to prescription drugs in the order, financial information associated with the order (for example, an invoice or an account statement), etc.

In some implementations, the literature device 628 folds or otherwise prepares the literature for inclusion with a prescription drug order (e.g., in a shipping container). In other implementations, the literature device 628 prints the literature and is separate from another device that prepares the printed literature for inclusion with a prescription order.

The packing device 626 packages the prescription order in preparation for shipping the order. The packing device 626 may box, bag, or otherwise package the fulfilled prescription order for delivery. The packing device 626 may further place inserts (e.g., literature or other papers, etc.) into the packaging received from the literature device 628. For example, bulk prescription orders may be shipped in a box, while other prescription orders may be shipped in a bag, which may be a wrap seal bag.

The packing device 626 may label the box or bag with an address and a recipient's name. The label may be printed and affixed to the bag or box, be printed directly onto the bag or box, or otherwise associated with the bag or box. The packing device 626 may sort the box or bag for mailing in an efficient manner (e.g., sort by delivery address, etc.). The packing device 626 may include ice or temperature sensitive elements for prescriptions that are to be kept within a temperature range during shipping (for example, this may be necessary in order to retain efficacy). The ultimate package may then be shipped through postal mail, through a mail order delivery service that ships via ground and/or air (e.g., UPS, FEDEX, or DHL, etc.), through a delivery service, through a locker box at a shipping site (e.g., AMAZON locker or a PO Box, etc.), or otherwise.

The unit of use packing device 630 packages a unit of use prescription order in preparation for shipping the order. The unit of use packing device 630 may include manual scanning of containers to be bagged for shipping to verify each container in the order. In an example implementation, the manual scanning may be performed at a manual scanning station. The pharmacy fulfillment device 512 may also include a mail manifest device 632 to print mailing labels used by the packing device 626 and may print shipping manifests and packing lists.

While the pharmacy fulfillment device 512 in FIG. 6 is shown to include single devices 606-632, multiple devices may be used. When multiple devices are present, the multiple devices may be of the same device type or models, or may be a different device type or model. The types of devices 606-632 shown in FIG. 6 are example devices. In other configurations of the system 500, lesser, additional, or different types of devices may be included.

Moreover, multiple devices may share processing and/or memory resources. The devices 606-632 may be located in the same area or in different locations. For example, the devices 606-632 may be located in a building or set of adjoining buildings. The devices 606-632 may be interconnected (such as by conveyors), networked, and/or otherwise in contact with one another or integrated with one another (e.g., at the high-volume fulfillment center, etc.). In addition, the functionality of a device may be split among a number of discrete devices and/or combined with other devices.

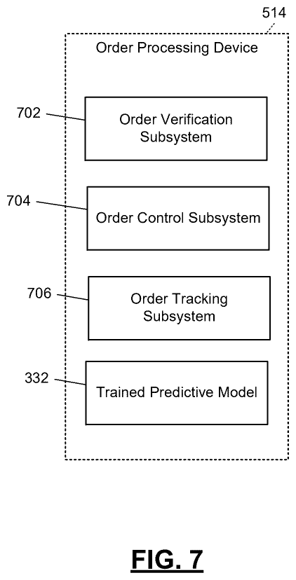

FIG. 7 illustrates the order processing device 514 according to an example implementation. The order processing device 514 may be used by one or more operators to generate prescription orders, make routing decisions, make prescription order consolidation decisions, track literature with the system 500, and/or view order status and other order related information. For example, the prescription order may be comprised of order components.

The order processing device 514 may receive instructions to fulfill an order without operator intervention. An order component may include a prescription drug fulfilled by use of a container through the system 500. The order processing device 514 may include an order verification subsystem 702, an order control subsystem 704, and/or an order tracking subsystem 706. Other subsystems may also be included in the order processing device 514.

The order verification subsystem 702 may communicate with the benefit manager device 502 to verify the eligibility of the member and review the formulary to determine appropriate copayment, coinsurance, and deductible for the prescription drug and/or perform a DUR (drug utilization review). Other communications between the order verification subsystem 702 and the benefit manager device 502 may be performed for a variety of purposes.

The order control subsystem 704 controls various movements of the containers and/or pallets along with various filling functions during their progression through the system 500. In some implementations, the order control subsystem 704 may identify the prescribed drug in one or more than one prescription orders as capable of being fulfilled by the automated dispensing device 614. The order control subsystem 704 may determine which prescriptions are to be launched and may determine that a pallet of automated-fill containers is to be launched.

The order control subsystem 704 may determine that an automated-fill prescription of a specific pharmaceutical is to be launched and may examine a queue of orders awaiting fulfillment for other prescription orders, which will be filled with the same pharmaceutical. The order control subsystem 704 may then launch orders with similar automated-fill pharmaceutical needs together in a pallet to the automated dispensing device 714. As the devices 606-632 may be interconnected by a system of conveyors or other container movement systems, the order control subsystem 704 may control various conveyors: for example, to deliver the pallet from the loading device 608 to the manual fulfillment device 616 from the literature device 628, paperwork as needed to fill the prescription.

The order tracking subsystem 706 may track a prescription order during its progress toward fulfillment. The order tracking subsystem 706 may track, record, and/or update order history, order status, etc. The order tracking subsystem 706 may store data locally (for example, in a memory) or as a portion of the order data 618 stored in the storage device 510.

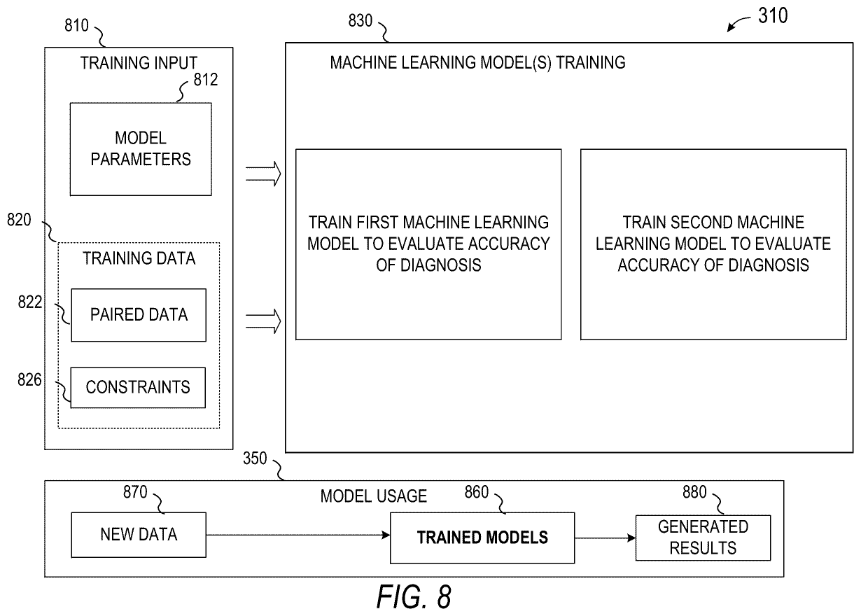

FIG. 8 is a block diagram of an example service of a predictive model server 310 that may be deployed within the systems and methods described herein. Training input 310 includes model parameters 812 and training data 820 (e.g., training data 320) which may include paired training data sets 822 (e.g., input-output training pairs) and constraints 826. Model parameters 812 stores or provides the parameters or coefficients of corresponding ones of machine learning models. During training, these parameters 812 are adapted based on the input-output training pairs of the training data sets 822. After the parameters 812 are adapted (after training), the parameters are used by trained models 860 to implement the trained machine learning models on a new set of data 870.

Training data 820 includes constraints 826 which may define the constraints of a given patient information features. The paired training data sets 822 may include sets of input-output pairs, such as pairs of a plurality of medicinal drug prescription features and features of entities associated with the medicinal drug prescriptions. The paired training data sets 822 may include sets of input-output pairs, such as pairs of a plurality of prescription entity features and features of medical signatures associated with the prescription entities. Some components of training input 810 may be stored separately at a different off-site facility or facilities than other components.

Machine learning model(s) training 830 trains one or more machine learning techniques based on the sets of input-output pairs of paired training data sets 822. For example, the model training 830 may train the machine learning (ML) model parameters 812 by minimizing a loss function based on one or more ground-truth data.

The ML models can include any one or combination of classifiers or neural networks, such as an artificial neural network, a convolutional neural network, an adversarial network, a generative adversarial network, a deep feed forward network, a radial basis network, a recurrent neural network, a long/short term memory network, a gated recurrent unit, an auto encoder, a variational autoencoder, a denoising autoencoder, a sparse autoencoder, a Markov chain, a Hopfield network, a Boltzmann machine, a restricted Boltzmann machine, a deep belief network, a deep convolutional network, a deconvolutional network, a deep convolutional inverse graphics network, a liquid state machine, an extreme learning machine, an echo state network, a deep residual network, a Kohonen network, a support vector machine, a neural Turing machine, and the like.

Particularly, a first ML model of the ML models can be applied to a training batch of medical records to evaluate accuracy of medical diagnosis on a subset of diagnosis that are considered critical illnesses. In some implementations, a derivative of a loss function is computed based on a comparison of the medical records and misdiagnosis and accurate diagnosis. The accurate diagnosis can be a ground truth for parameters of the first ML model and can be updated based on the computed derivative of the loss function. The result of minimizing the loss function for multiple sets of training data trains, adapts, or optimizes the model parameters 812 of the corresponding first ML model. In this way, the first ML model is trained to establish a relationship between a plurality of training medicinal diagnosis, accurate in critical diagnosis.

A second ML model of the ML models can be applied to a training batch of further medical records features to estimate or generate a prediction of the medical diagnosis for critical illnesses and diagnosis.

After the machine learning models are trained, new data 870, including one or more medicinal drug prescription features are received and/or derived from a further data in the data storage 510. The first trained machine learning model may be applied to the new data 870 to generate results 880 including a prediction of one or more vectors for the model.

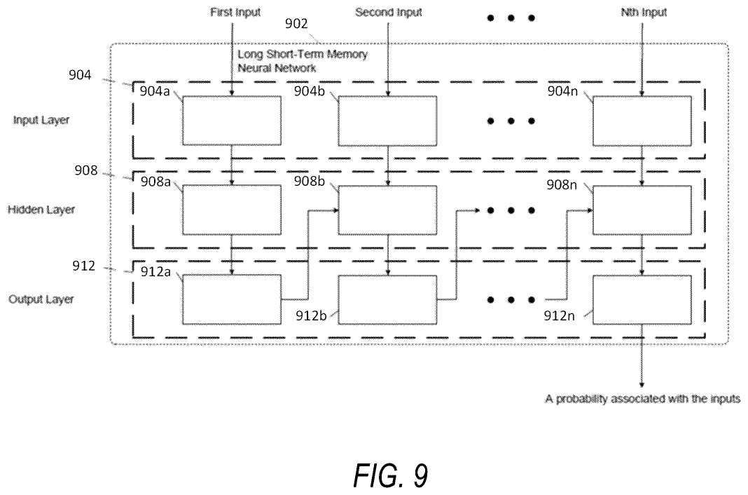

FIG. 9 is a functional block diagram of an example neural network 902 that can be used for the inference engine or other functions (e.g., engines) as described herein to produce a predictive model. The predictive model can evaluate diagnosis of critical illness and process the described herein. In an example, the neural network 902 can be a LSTM neural network. In an example, the neural network 902 can be a recurrent neural network (RNN). The example neural network 902 may be used to implement the machine learning as described herein, and various implementations may use other types of machine learning networks. The neural network 902 includes an input layer 904, a hidden layer 908, and an output layer 912. The input layer 904 includes inputs 904a, 904b. . . 904n. The hidden layer 908 includes neurons 908a, 908b. . . 908n. The output layer 912 includes outputs 912a, 912b. . . 912n.

Each neuron of the hidden layer 908 receives an input from the input layer 904 and outputs a value to the corresponding output in the output layer 912. For example, the neuron 908a receives an input from the input 904a and outputs a value to the output 912a. Each neuron, other than the neuron 908a, also receives an output of a previous neuron as an input. For example, the neuron 908b receives inputs from the input 904b and the output 912a. In this way the output of each neuron is fed forward to the next neuron in the hidden layer 908. The last output 912n in the output layer 912 outputs a probability associated with the inputs 904a-904n. Although the input layer 904, the hidden layer 908, and the output layer 912 are depicted as each including three elements, each layer may contain any number of elements. Neurons can include one or more adjustable parameters, weights, rules, criteria, or the like.

In various implementations, each layer of the neural network 902 must include the same number of elements as each of the other layers of the neural network 902. For example, training features (e.g., medical data associated with a critical illness) may be processed to create the inputs 904a-904n.

The neural network 902 may implement a first model to produce models based on medical data. More specifically, the inputs 904a-904n can include fields of the prescription as data features (binary, vectors, factors or the like) stored in the storage device 110. The features of the prescription can be provided to neurons 908a-908n for analysis and connections between the known facts. The neurons 908a-908n, upon finding connections, provides the potential connections as outputs to the output layer 912, which determines a set of entities associated with the prescription.

The neural network 902 may implement a second model to produce one or more medical models with critical illnesses. More specifically, the inputs 904a-904n can include data associates with members, illness, clinical data, and past diagnosis, determined by the first model as data features (binary, vectors, factors or the like) stored in the storage device 110. The features of the model can be provided to neurons 908a-908n for analysis and connections between the known facts. The neurons 908a-908n, upon finding connections, provides the potential connections as outputs to the output layer 912, which determines where a diagnosis meets an accuracy threshold to control one or more medical treatments or flag a diagnosis record for further intervention.

The neural network 902 can perform any of the above calculations. The output of the neural network 902 can be used to trigger display of a prompt that includes the accuracy prediction to an operator in a GUI on a display device. For example, the prompt (e.g., notification) can be provided to a device associated with a PBM, health plan manager, pharmacy, physician, caregiver, and/or a patient. The prompt can include the original diagnosis received from the healthcare professional, the predicted model output, a confidence level associated with the predicted model output.

In some examples, a convolutional neural network may be implemented. Similar to neural networks, convolutional neural networks include an input layer, a hidden layer, and an output layer. However, in a convolutional neural network, the output layer includes one fewer output than the number of neurons in the hidden layer and each neuron is connected to each output. Additionally, each input in the input layer is connected to each neuron in the hidden layer. In other words, input 904a is connected to each of neurons 908a, 908b. . . 908n.

Example systems and methods for using trained predictive modeling to reduce misdiagnoses of critical illnesses and control medical devices, e.g., pharmacy devices, chemotherapy devices, genetic therapy devices, stem cell implant devices, radiation devices and the like, are described herein and illustrated in the accompanying drawings. This written description uses examples to disclose aspects of the disclosure and also to enable a person skilled in the art to practice the aspects, including making or using the above-described systems and executing or performing the above-described methods. Examples described herein ensure predictive accuracy and facilitate providing improved workflows, runtime performance, and data quality, as well as assist in reducing medication errors or over prescribing. By identifying cases which are likely to benefit from a clinical consultation (or second opinion) review prior to treatment (e.g., dispensing of medications that may result in side effects), the risk of improper, unwarranted, and/or missed treatment can be mitigated, and the quality and cost of healthcare can be significantly improved.

The foregoing description is merely illustrative in nature and is in no way intended to limit the disclosure, its application, or uses. The systems and methods improve cancer treatments, e.g., medication dispensing, reduce unnecessary surgery, chemotherapy, radiation, immunotherapy, targeted therapy, hormone therapy, stem cell transplants, and emerging treatments such as gene and cell-based therapies, The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

When introducing elements of the disclosure or the examples thereof, the articles “a,” “an,” “the,” and “said” are intended to mean that there are one or more of the elements. References to an “embodiment” or an “example” of the present disclosure are not intended to be interpreted as excluding the existence of additional embodiments or examples that also incorporate the recited features. As used herein, the phrase at least one of A, B, and C should be construed to mean a logical (A OR B OR C), using a non-exclusive logical OR, and should not be construed to mean “at least one of A, at least one of B, and at least one of C.”

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements.

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A. The term subset does not necessarily require a proper subset. In other words, a first subset of a first set may be coextensive with (equal to) the first set.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave). The term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A trained predictive server for improving claim data processing comprising:
a memory;
a processor configured to:
train, with a machine learning system, a model to assess diagnostic or treatment determinations to generate a trained predictive model;
receive, from a first claim database server, a set of prior authorization (PA) data associated with a medical claim for a patient, wherein the medical claim is associated with a first provider;
determine that the set of PA data indicates that the medical claim is associated with a qualifying critical illness;
extract, with a machine learning model, component data from the set of PA data by extracting features comprising one or more of diagnostic data, treatment data, patient demographic data, patient geographic data, patient socioeconomic data, healthcare provider data, or healthcare provider reputation data from the set of PA data to store as the component data;
apply the extracted component data to the trained predictive model that is associated with the qualifying critical illness to determine a likelihood of whether the medical claim is associated with an inaccurate diagnosis and treatment plan;
compare the likelihood against a plurality of predetermined thresholds, the plurality of predetermined thresholds including an upper predetermined threshold and a lower predetermined threshold;
on condition that the likelihood is less than the lower predetermined threshold, automatically approve and process the medical claim;
on condition that the likelihood is between the upper predetermined threshold and the lower predetermined threshold;
determine that the medical claim is not yet approved and avoid processing the medical claim based on the medical claim not yet being approved; and
transmit an alert to a second device to request additional input from a second provider regarding whether a consulting review should be performed on the medical claim or whether the medical claim is approved for processing;
on condition that the likelihood is greater than the upper predetermined threshold, determine that the medical claim is inaccurate, flag the medical claim to halt use of medical devices associated with the medical claim and collect further data associated with one or more of the medical claim or the inaccurate diagnosis and treatment plan from one or more of the first claim database server or a first data warehouse server, wherein the further data includes one or more of medical records, imaging, or laboratory results, identify, from the one or more of the first claim database server or the first data warehouse server, a third provider from a list of candidate consulting review providers that is different from the first provider, and initiate a second review of the medical claim by the third provider by providing the further data to the third provider; and
on condition that the likelihood is less than the upper predetermined threshold, use a medical device to treat the medical condition associated with the medical claim.

2. The trained predictive server of claim 1, wherein the processor is further configured to:
identify a list of qualifying critical illnesses from a storage device in communication with the trained predictive server; and
apply the list to the set of PA data to determine that the set of PA data indicates that the medical claim is associated with the qualifying critical illness.

3. The trained predictive server of claim 1, wherein the processor is further configured to:
identify a list of the features for extraction; and
extract the component data, based on the list of features, from the set of PA data.

4. The trained predictive server of claim 1, wherein the processor is further configured to identify the second provider based on the set of PA data.

5. The trained predictive server of claim 1, wherein use the medical device includes authorizing an automated pharmacy to fulfill a prescription.

6. The trained predictive server of claim 5, wherein authorizing an automated pharmacy to fulfill a prescription includes moving a container to a pill dispenser and automatically dispensing pills into the container with the pills being prescribed as part of the treatment for the medical diagnosis.

7. The trained predictive server of claim 6, wherein to identify, from the one or more of the first claim database server or the first data warehouse server, the third provider from the list of candidate consulting review providers, the processor is further configured to:
identify the third provider from the list of candidate consulting review providers based on candidate provider location data indicating that the third provider is geographically diverse from the first provider and the patient.

8. The trained predictive server of claim 1, wherein the processor is further configured to: conduct a correlation analysis between historical PA data and the features based on a correlation coefficient between the features and result data describing or indicating an adverse outcome; and determine that the features are to be extracted from the set of PA data based on the correlation analysis.

9. A method performed by a trained predictive server including a processor and a memory for improved claim data processing, the method comprising:
training, with a machine learning system, a model to assess diagnostic or treatment determinations to generate a trained predictive model;
receiving, from a first claim database server, a set of prior authorization (PA) data associated with a medical claim for a patient, wherein the medical claim is associated with a first provider;
determining that the set of PA data indicates that the medical claim is associated with a qualifying critical illness;
extracting, with a machine learning model, component data from the set of PA data by extracting features comprising one or more of diagnostic data, treatment data, patient demographic data, patient geographic data, patient socioeconomic data, healthcare provider data, or healthcare provider reputation data from the set of PA data to store as the component data;
applying the extracted component data to the trained predictive model that is associated with the qualifying critical illness to determine a likelihood of whether the medical claim is associated with an inaccurate diagnosis and treatment plan;
compare the likelihood against a plurality of predetermined thresholds, the plurality of predetermined thresholds including an upper predetermined threshold and a lower predetermined threshold;
on condition that the likelihood is less than the lower predetermined threshold, automatically approving and processing the medical claim;
on condition that the likelihood is between the upper predetermined threshold and the lower predetermined threshold;
determining that the medical claim is not yet approved and avoid processing the medical claim based on the medical claim not yet being approved;
flag the medical claim to halt use of medical devices associated with the medical claim,
transmitting an alert to a second device to request additional input from a second provider regarding whether a consulting review should be performed on the medical claim or whether the medical claim is approved for processing;
on condition that the likelihood is greater than the upper predetermined threshold, determining that the medical claim is inaccurate, collecting further data associated with one or more of the medical claim or the inaccurate diagnosis and treatment plan from one or more of the first claim database server or a first data warehouse server, wherein the further data includes one or more of medical records, imaging, or laboratory results, identifying, from the one or more of the first claim database server or the first data warehouse server, a third provider from a list of candidate consulting review providers that is different from the first provider, and initiating a second review of the medical claim by the third provider by providing the further data to the third provider; and
on condition that the likelihood is less than the upper predetermined threshold, operating a medical device to treat the medical condition associated with the medical claim.

10. The method of claim 9, further comprising identifying a list of qualifying critical illnesses from a storage device in communication with the trained predictive server, wherein the list of qualifying critical illnesses is applied to determine whether the set of PA data indicates that the medical claim is associated with the qualifying critical illness.

11. The method of claim 9, further comprising identifying a list of the features for extraction, wherein the list of features for extraction is used to extract the component data from the set of PA data.

12. The method of claim 9, further comprising: identifying the second provider based on the set of PA data.

13. The method of claim 9, wherein the identifying the third provider comprises identifying the third provider from the list of candidate consulting review providers based on candidate provider location data indicating that the third provider is geographically diverse from the first provider and the patient.

14. The method of claim 9, further comprising:
conducting a correlation analysis between historical PA data and the features based on a correlation coefficient between the features and result data describing or indicating an adverse outcome; and
determining that the features are to be extracted from the set of PA data based on the correlation analysis.

15. A trained predictive system for improving claim data processing comprising:
a first claim database server comprising a database processor and a database memory, the database memory includes a set of prior authorization (PA) data associated with a medical claim for a patient, the database processor configured to determine that the set of PA data indicates that the medical claim is associated with a qualifying critical illness;
a first data warehouse server comprising a warehouse processor and a warehouse memory, the warehouse memory including a plurality of historical PA data associated with one or more of a plurality of patients or healthcare providers, and a plurality of result data associated with the plurality of historical PA data; and
a trained predictive server in communication with the first claim database server and the first data warehouse server, the trained predictive server comprising a predictive processor and a predictive memory, the predictive processor configured to:
train, with a machine learning system, a model to assess diagnostic or treatment determinations to generate a trained predictive model;
receive, from the first claim database server, the set of PA data associated with the medical claim for the patient, wherein the medical claim is associated with a first provider;
extract, with a machine learning model, component data from the set of PA data by extracting features comprising one or more of diagnostic data, treatment data, patient demographic data, patient geographic data, patient socioeconomic data, healthcare provider data, or healthcare provider reputation data from the set of PA data to store as the component data;
apply the extracted component data to the trained predictive model that is associated with the qualifying critical illness to determine a likelihood of whether the medical claim is associated with an inaccurate diagnosis and treatment plan;
compare the likelihood against a plurality of predetermined thresholds, the plurality of predetermined thresholds including an upper predetermined threshold and a lower predetermined threshold;
on condition that the likelihood is less than the lower predetermined threshold, automatically approve and process the medical claim, and flag the medical claim record to allow use of medical devices;
on condition that the likelihood is between the upper predetermined threshold and the lower predetermined threshold, determine that the medical claim is not yet approved and avoid processing the medical claim based on the medical claim not yet being approved and flag the medical claim record to not allow use of medical devices for the medical claim; and
transmit an alert to a second device to request additional input from a second provider regarding whether a consulting review should be performed on the medical claim or whether the medical claim is approved for processing;
on condition that the likelihood is greater than the upper predetermined threshold, determine that the medical claim is inaccurate, flag the medical claim record to prevent use of medical devices for treatment of the medical diagnosis and collect further data associated with one or more of the medical claim or the inaccurate diagnosis and treatment plan from one or more of the first claim database server or the first data warehouse server, wherein the further data includes one or more of medical records, imaging, or laboratory results, identify, from the one or more of the first claim database server or the first data warehouse server, a third provider from a list of candidate consulting review providers that is different from the first provider, and initiate a second review of the medical claim by the third provider by providing the further data to the third provider; and
control use of medical devices based on the flagged status of the medical claim record.

16. The trained predictive system of claim 15, wherein the database processor is further configured to:
receive a list of qualifying critical illnesses from the first data warehouse server; and
apply the list to the set of PA data to determine that the set of PA data indicates that the medical claim is associated with the qualifying critical illness.

17. The trained predictive system of claim 15, wherein the processor is further configured to:
identify a list of the features for extraction; and
extract the component data, based on the list of features, from the set of PA data.

18. The trained predictive system of claim 15, wherein the database processor is further configured to identify the second provider based on the set of PA data.

19. The trained predictive system of claim 15, wherein to identify, from the one or more of the first claim database server or the first data warehouse server, the third provider from the list of candidate consulting review providers, the predictive processor is further configured to: identify the third provider from the list of candidate consulting review providers based on candidate provider location data indicating that the third provider is geographically diverse from the first provider and the patient.

20. The trained predictive system of claim 15, wherein the predictive processor is further configured to:
conduct a correlation analysis between historical PA data and the features based on a correlation coefficient between the features and result data describing or indicating an adverse outcome; and
determine that the features are to be extracted from the set of PA data based on the correlation analysis.
